(19) United States
(12) Patent Application Publication
(10) Pub. No.: US 2026/0272798 A1
(43) Pub. Date: Sep. 17, 2026

(54) Formulations and Applications of Feruloyl Glycerides

(71) Applicant: Midwest Bioprocessing Center, LLC, Peoria, IL (US)

(72) Inventor: Mark Chandler, Kirkwood, DE (US)

(21) Appl. No.: 19/469,003

(22) PCT Filed: Mar. 28, 2024
(86) PCT No.: PCT/US2024/022040

§ 371 Date: Sep. 25, 2025

Related U.S. Application Data
(60) Provisional application No. 63/492,643, filed on Mar. 28, 2023.

(51) Int. Cl. A61K 8/37; A61K 8/25; A61K 8/27; A61K 8/29; A61Q 17/04

(52) U.S. Cl. CPC A61K 8/375; A61K 8/25; A61K 8/27; A61K 8/29; A61Q 17/04

(57) ABSTRACT

Formulations with feruloyl glycerides yield novel enhancements to improve SPF of sunscreens, offer enhanced protection of active ingredients and therapeutic agents from UV light and serve as an antioxidant. These agents have the advantage of being synthesized from natural materials, while providing a value-added use for vegetable oils. They are readily applied to sunscreen, lotion, haircare, suncare, personal care, pharmaceutical and industrial formulations.

### CROSS REFERENCE TO RELATED APPLICATIONS

This application claims the benefit of U.S. Provisional Application No. 63/492,643, filed Mar. 28, 2023, which is incorporated by reference herein in its entirety.

### BACKGROUND OF THE DISCLOSURE

### Field of Invention

This disclosure relates to sunscreen, lotion, haircare, suncare, personal care, pharmaceutical, and industrial formulations enhanced with feruloyl glycerides.

### Technical Background

Ultraviolet (UV) radiation is one of the major environmental factors that affect skin health and can cause damage and changes to industrial materials. Exposure to UV radiation causes various forms of skin damage, including sunburn, photoaging, and skin cancer. Therefore, it is essential to protect the skin from UV radiation using appropriate measures, including the use of sunscreens. Sunscreens contain UV filters that absorb, reflect, or scatter UV radiation, preventing it from reaching the skin. In addition to protecting the skin, UV filters also protect active ingredients in cosmetics and topical therapeutics from photodegradation.

There are a number of challenges associated with properly formulating sunscreens and topical products. Physical (inorganic) sunscreens such as Zinc Oxide (ZnO) and Titanium Dioxide (TiO₂) are difficult to formulate by themselves at high SPF (sun protection factor) and can leave a white cast on the skin. Organic (chemical) sunscreens and UV filters can aid in improving formulation and SPF, but many of these provide modest SPF protection and are falling out of favor with the FDA due to health and environmental concerns. Thus, there is a need for new biobased molecules and formulations which can increase SPF and/or otherwise enhance and protect skin and other active ingredients from the harmful effect of UV exposure. Such formulations would be useful in a wide range of applications such as sunscreens, suncare products, lotions, haircare and skincare products, other personal care products, pharmaceutical formulations, and industrial applications including textiles, materials, and others.

### SUMMARY OF THE DISCLOSURE

This disclosure describes compositions and materials containing SPF boosters.

In a first aspect, the present disclosure provides a composition that includes
a) at least one sunscreen active agent; and
b) a compound of formula I:

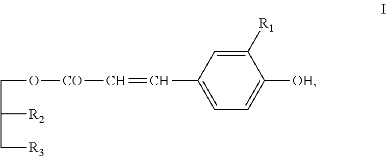

wherein R₁ is OCH₃ or H, and
wherein R₂ and R₃ are each independently selected from OH and a C2-C24 fatty acid moiety.

In one embodiment of the first aspect, the at least one sunscreen active agent is a physical sunscreen comprising zinc oxide or titanium dioxide.

In one embodiment of the first aspect, the at least one sunscreen active agent is an organic sunscreen comprising one or more of homosalate, octisalate, octocrylene, avobenzone, anthranilates, cinnamic derivatives, dibenzoylmethane derivatives, salicylic derivatives, camphor derivatives, triazine derivatives, benzophenone derivatives, B,β-diphenylacrylate derivatives, benzo-benzalmalonate benzimidazole derivatives, imidazolines, bisbenzoazolyl derivatives, p-aminobenzoic acid (PABA) derivatives, meth-ylenebishydroxyphenylbenzotriazole) derivatives, screening polymers and screening silicones, dimers derived from a-alkylstyrene, 4,4-diarylbutadienes and their mixtures.

In one embodiment of the first aspect or previous embodiments thereof, the combination of the at least one sunscreen active agent and the compound provide an SPF greater than the additive properties of the individual agents.

In one embodiment of the first aspect or previous embodiments thereof, the composition has an in vitro SPF of at least 10.

In one embodiment of the first aspect or previous embodiments thereof, the combination is combined with a reduced amount of physical sunscreen to the point of yielding a formulation that is transparent and free of white cast effect while retaining high SPF.

In a second aspect, the present disclosure provides a composition that includes
a) at least one sunscreen active agent;
b) a porous micro silica; and
c) a compound of formula I:

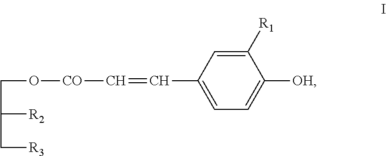

wherein R₁ is OCH₃ or H, and
wherein R₂ and R₃ are each independently selected from OH and a C2-C24 fatty acid moiety.

In one embodiment of the second aspect, the combination of ingredients is combined with at least one physical or organic sunscreen.

In one embodiment of the second aspect or previous embodiment thereof, the combination of ingredients provides an SPF greater than the additive properties of the individual agents.

In one embodiment of the second aspect, the at least one physical sunscreen is one or more of zinc oxide or titanium dioxide.

In one embodiment of the second aspect, the at least one organic sunscreen is one or more of homosalate, octisalate, octocrylene, avobenzone, anthranilates, cinnamic derivatives, dibenzoylmethane derivatives, salicylic derivatives, camphor derivatives, triazine derivatives, benzophenone derivatives, B,β-diphenylacrylate derivatives, benzo-benzalmalonate benzimidazole derivatives, imidazolines, bisbenzoazolyl derivatives, p-aminobenzoic acid (PABA) derivatives, meth-ylenebishydroxyphenylbenzotriazole) derivatives, screening polymers and screening silicones, dimers derived from a-alkylstyrene, 4,4-diarylbutadienes and their mixtures.

In one embodiment of the second aspect or previous embodiments thereof, the composition has an in vitro SPF of at least 10.

In a third aspect, the present disclosure provides a composition that includes
a) an optional solvent;
b) an optional humectant;
c) an optional base;
d) a feruloyl glyceride;
e) an optional emollient;
f) an optional detergent;
g) an optional polymer;
h) an optional emulsifier;
i) an optional micro silica
j) an optional active; and
k) an optional sunscreen.

In one embodiment of the third aspect, the composition includes
a) about 80-99.9% solvent; and
b) about 0.1% to about 20% of a feruloyl glyceride.

In one embodiment of the third aspect, the composition includes
a) about 0.1% to about 20% of a feruloyl glyceride; and
b) about 1% to about 25% of a sunscreen.

In one embodiment of the third aspect, the composition includes
a) about 0.1% to about 20% of a feruloyl glyceride;
b) about 1% to about 25% of a sunscreen; and
c) about 0.5% to about 20% of a micro silica.

In one embodiment of the third aspect or previous embodiments thereof, the sunscreen is a physical sunscreen comprising zinc oxide and/or titanium dioxide.

In one embodiment of the third aspect or previous embodiments thereof, the sunscreen is an organic sunscreen comprising one or more of homosalate, octisalate, octocrylene, avobenzone, anthranilates, cinnamic derivatives, dibenzoylmethane derivatives, salicylic derivatives, camphor derivatives, triazine derivatives, benzophenone derivatives, B,β-diphenylacrylate derivatives, benzo-benzalmalonate benzimidazole derivatives, imidazolines, bisbenzoazolyl derivatives, p-aminobenzoic acid (PABA) derivatives, meth-ylenebishydroxyphenylbenzotriazole) derivatives, screening polymers and screening silicones, dimers derived from a-alkylstyrene, 4,4-diarylbutadienes and their mixtures.

In one embodiment of the third aspect, the composition includes
a) about 0.1% to about 20% of a feruloyl glyceride; and
b) an active ingredient comprising one or more of a retinoid, hyaluronic acid, vitamin C or a derivative thereof, vitamin D or derivative thereof, vitamin E or derivative thereof, a tocopherol, a bakuchiol, or a photosensitive active ingredient.

In a fourth aspect, the present disclosure provides a topical composition that include a compound of formula I:

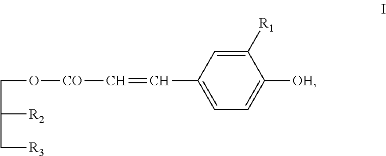

wherein R₁ is OCH₃ or H, and
wherein R₂ and R₃ are each independently selected from OH and a C2-C24 fatty acid moiety and the calculated active skin gap (AKG) is greater than 8 and Mvol is greater than 500 indicating that the molecule does not penetrate the skin.

In one embodiment of the fourth aspect, the composition includes about 0.1% to about 20% of a feruloyl glyceride.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1. SPF Boost with Combinations of Feruloyl glycerides, ZnO and silica spheres. Scatter plot show that when either feruloyl coconut glycerides or feruloyl soy glycerides are combined with a mixture of ZnO and/or micro silica they have a synergistic boost in the overall SPF of the formulation.

FIG. 2. SPF Boost. Bar chart similarly showing that when either feruloyl coconut glycerides or feruloyl soy glycerides are combined with a mixture of ZnO and/or porous micro silica they have a synergystic boost in the overall SPF of the formulation.

FIG. 3. SPF Boost with 5% feruloyl glycerides and 10% Inorganic Sunscreen. Combining feruloyl glycerides with an inorganic sunscreen such as ZnO gives a synergistic boost of in vitro SPF levels.

### DETAILED DESCRIPTION

### Overview

This disclosure relates to feruloyl-substituted and coumaryl-substituted acylglycerols, their formulation, and use to enhance SPF or UV resistance in cosmetic, personal care, textile, and industrial products. The present disclosure relates to a novel formulation for sunscreens, for example, that provides superior protection against ultraviolet (UV) radiation. The formulations combine the benefits of feruloyl-substituted and coumaryl-substituted acylglycerols (feruloyl glycerides) with various UV filters, SPF boosters, porous microsilica, and other ingredients to achieve a higher sun protection factor (SPF). The addition of feruloyl glycerides has now been shown to substantially boost the SPF and efficacy when combined with sunscreens such as ZnO, TiO₂, organic sunscreens, and likely other sunscreen molecules as well to levels that could not previously have been predicted.

UV filters are essential ingredients in sunscreens that absorb or reflect UV radiation and prevent it from penetrating the skin and can also be used in a variety of industrial applications to protect or modify changes in materials due to UV exposure. Different UV filters have different properties and limitations, such as their ability to block specific wavelengths of UV radiation, their photostability, their potential to cause skin irritation or allergies, and their compatibility with other ingredients. Therefore, selecting and combining the right UV filters in a sunscreen formulation is crucial to achieving the desired SPF, spectrum, and performance.

SPF boosters are additional ingredients that can enhance the UV protection of a sunscreen (and other compositions and materials) by increasing the amount of UV radiation that it can absorb or reflect. SPF boosters can also improve the stability, spreadability, or water resistance of the sunscreen. SPF boosters do not necessarily have significant SPF properties on their own.

Microsilica, also known as fumed silica, is a fine powder that can be added to sunscreens to improve their texture, reduce their greasiness, and enhance their water resistance. Microsilica can form a network of particles that stabilizes the emulsion and prevents the sunscreen from separating or leaking. Microsilica can also improve the adhesion of the sunscreen to the skin and provide a soft, velvety feel. Additionally, porous micro silica can offer advantages to formulations over standard microsilica.

Feruloyl-substituted and coumaryl-substituted acylglycerols including feruloyl glycerides are molecules that combine the UV spectrum and antioxidant properties of ferulic acid with the formulation advantages of triglycerides such as soybean oil, coconut oil, hemp seed oil, safflower oil, jojoba oil, shea butter, and a variety of other natural or synthetic triglycerides and vegetable oils.

The present disclosure provides, for example, effective sunscreen formulations that combines the benefits of feruloyl-substituted and coumaryl-substituted acylglycerols (feruloyl glycerides) with the benefits of various UV filters, SPF boosters, microsilica to achieve a high SPF and broad-spectrum protection. The formulation can be applied to various types of skin and can provide long-lasting and effective sun protection.

When feruloyl glycerides were combined with either sunscreen actives alone or in combination with porous microsilica particles it was found that a significant and surprising boost in in SPF was achieved when formulations were tested with an in vitro SPF test.

The following description provides further details and examples of the disclosure.

### INTRODUCTION

Ultraviolet (UV) radiation is one of the major environmental factors that affect skin health and can cause damage and changes to industrial materials. Exposure to UV radiation causes various forms of skin damage, including sunburn, photoaging, and skin cancer. Therefore, it is essential to protect the skin from UV radiation using appropriate measures, including the use of sunscreens. Sunscreens contain UV filters that absorb, reflect, or scatter UV radiation, preventing it from reaching the skin. In addition to protecting the skin, UV filters also protect active ingredients in cosmetics and topical therapeutics from photodegradation.

There are a number of challenges associated with properly formulating sunscreens and topical products. Physical (inorganic) sunscreens such as Zinc Oxide (ZnO) and Titanium Dioxide (TiO₂) are difficult to formulate by themselves at high SPF and can leave a white cast on the skin. Organic (chemical) sunscreens and UV filters can aid in improving formulation and SPF but many of these provide modest SPF protection and are falling out of favor with the FDA due to health and environmental concerns. Thus, described herein are for new biobased molecules and formulations that enhance and protect skin and other active ingredients from the harmful effect of UV exposure. These formulations are useful in a wide range of applications such as sunscreens, suncare products, lotions, haircare and skincare products, other personal care products, pharmaceutical formulations, and industrial applications including textiles, materials, and others.

### The Importance of UV Protection

UV Radiation. UV radiation is classified into three types based on wavelength: UVA (320-400 nm), UVB (290-320 nm), and UVC (100-290 nm). UVC radiation is absorbed by the ozone layer and does not reach the earth's surface. UVA and UVB radiation are the primary causes of skin damage. UVA radiation penetrates deep into the skin and causes sunburn, skin cancer, premature photoaging, wrinkling, and loss of elasticity. UVA radiation also contributes to the development of skin cancer. UVB radiation primarily affects the superficial layers of the skin and causes sunburn, erythema, and DNA damage. UVB radiation is also a major cause of skin cancer.

Sunscreen products are designed to protect the skin from the harmful effects of ultraviolet (UV) radiation. Sun protection factor (SPF) is a measure of how well a sunscreen product protects the skin from UV radiation. The higher the SPF, the greater the protection. The formulations of sun care and sunscreen products play a critical role in increasing SPF.

Sunscreen products work by absorbing, reflecting, or scattering UV radiation. The active ingredients in sunscreens are either organic or inorganic. Organic ingredients, such as avobenzone and oxybenzone, and others absorb UV radiation and convert it into heat. Inorganic ingredients, such as zinc oxide and titanium dioxide, reflect and scatter UV radiation. Sunscreen products can often contain a combination of organic and inorganic ingredients along with other active ingredients, antioxidants, and molecules to improve their function. Organic sunscreen active agents include a variety of compounds such as anthranilates, cinnamic derivatives, dibenzoylmethane derivatives, salicylic derivatives, camphor derivatives, triazine derivatives, benzophenone derivatives, B,β-diphenylacrylate derivatives, benzo-benzalmalonate benzimidazole derivatives, imidazolines, bisbenzoazolyl derivatives, p-aminobenzoic acid (PABA) derivatives, meth-ylenebishydroxyphenylbenzotriazole) derivatives, screening polymers and screening silicones, dimers derived from a-alkylstyrene, 4,4-diarylbutadienes and their mixtures.

Not only is UV protection important for the skin but active ingredients used in cosmetic products and therapeutic molecules used in topical applications are frequently UV sensitive. UV radiation affects the stability of topical active compounds applied to the skin through oxidation and radical degradation. This same damaging property also effects topically applied active ingredients against acne, psoriasis, and dermatitis which face photodegradation from UV. Important vitamins and antioxidants such as vitamin A/Retinoids [1,2], vitamin C [3] and Vitamin E [4,5] undergo degradation when exposed to sunlight. Glucocorticosteroids are often UV sensitive molecules, with betamethasone, hydrocortisone and their derivatives as well-studied examples. [6-9] Topical antifungals such as itraconazole and fluconazole have shown to undergo photodegradation [10, 11], along with the anti-arthritic NSAIDs including piroxicam and naproxen. [12-14] Retinoids such as tretinoin/isotretinoin [15-17] and adapalene [16, 18] show breakdown after sunlight exposure, reducing their effectiveness. Thus UV protection is critical to maintain their efficacy.

It is envisioned that the compounds of this disclosure may also have certain industrial applications, such as a UV protectant for epoxies, paints, and other consumer products. For these applications, the compounds could either be formulated into the material to be protected, such as by blending into a paint, or they could be applied as a separate coating.

### Ingredients Used to Provide UV Protection in Sunscreens and Other Product.

There are two main types of UV filters used in sunscreens: physical and organic sunscreens. Physical sunscreens, such as zinc oxide (ZnO) and titanium dioxide (TiO₂), work by reflecting and scattering UV radiation. Organic sunscreens, also known as chemical sunscreens, contain organic compounds that absorb UV radiation and convert it into heat.

Physical Sunscreens such as ZnO and TiO₂. ZnO and TiO₂ are widely used in sunscreens due to their broad-spectrum UV protection and high photostability. However, formulating ZnO and TiO₂ in sunscreens presents several challenges, including the formation of a white cast and poor dispersion. A number of strategies can be employed in order to improve their performance including the following:

Particle Size Adjustment. The particle size of ZnO and TiO₂ affects their efficacy and appearance. Smaller particle sizes provide better UV protection but can increase the risk of skin irritation and phototoxicity. Larger particle sizes can reduce the efficacy of the UV filter and result in a white cast.

Surface Treatments. Surface treatments can improve the dispersion and reduce the white cast of ZnO and TiO₂. Common surface treatments include silica, alumina, and dimethicone. Surface treatments can also improve the photostability of ZnO and TiO₂.

Mixtures of Different Particle Sizes. Mixtures of different particle sizes can improve the performance and appearance of ZnO and TiO₂. The combination of smaller and larger particles can provide broad-spectrum UV protection and reduce the white cast.

Addition of Dispersion Aids. Dispersion aids, such as surfactants and polymers, can improve the dispersion of ZnO and TiO₂ in sunscreens. Surfactants can reduce the surface tension between the particles and the vehicle, allowing for better dispersion. Polymers can improve the stability and prevent settling of the particles.

While these methods all give incremental improvement, mixing the physical sunscreens with organic sunscreens, UV-filters, or other molecules are usually employed to help in their formulation and efficacy.

Organic (Chemical) Sunscreens and UV-Filters. Organic sunscreens contain organic compounds that absorb UV radiation and convert it into heat. UV filters and SPF boosters are used in combination to provide high SPF protection in sunscreens. SPF boosters enhance the UV protection provided by the UV filters and can improve the appearance and texture of the sunscreen. Common organic sunscreens can also include avobenzone, octinoxate, octisalate, homosalate, and oxybenzone. Organic sunscreen active agents include a variety of compounds such as anthranilates, cinnamic derivatives, dibenzoylmethane derivatives, salicylic derivatives, camphor derivatives, triazine derivatives, benzophenone derivatives, B,β-diphenylacrylate derivatives, benzo-benzalmalonate benzimidazole derivatives, imidazolines, bisbenzoazolyl derivatives, p-aminobenzoic acid (PABA) derivatives, meth-ylenebishydroxyphenylbenzotriazole) derivatives, screening polymers and screening silicones, dimers derived from a-alkylstyrene, 4,4-diarylbutadienes and their mixtures.

In recent years, there has been a shift away from existing UV filters and organic sunscreens in the United States due to concerns over their safety and effectiveness. The US Food and Drug Administration (FDA) has taken a more critical stance on these types of sunscreens and has proposed new regulations that could limit their use [19-24].

The FDA has been taking a more critical approach to UV filters and organic sunscreens, citing concerns over their safety and effectiveness. In February 2019, the FDA proposed new regulations that would require additional safety testing for 12 common UV filters, including oxybenzone and octinoxate. In addition to safety concerns, the FDA has also expressed doubts about the effectiveness of some UV filters and organic sunscreens. The agency has suggested that some of these products may not provide adequate protection against certain types of UV radiation, such as UVA1, which can cause skin damage and aging.

As a result most existing organic sunscreens and UV filters are falling out of favor with the FDA due to safety concerns. Oxybenzone has been linked to hormonal disruption and allergic reactions. Octinoxate and homosalate have been shown to accumulate in breast milk and have been linked to hormone disruption. Avobenzone and octocrylene have been shown to degrade into potentially harmful compounds upon exposure to UV radiation.

### Newer UV Filters and Other Methods to Boost SPF and Improve Sunscreen Performance

Ferulic acid and Feruloyl Glycerides. Hydroxycinnamic acids are naturally produced molecules found in the lignin and cellulose of plants. They are effective antioxidants and have broad UV absorption bands that are effective in both the UVA and UVB ranges, with a peak in the important UVA1 range. Ferulic acid (3-methoxy-4-hydroxycinnamic acid) is a protective molecule found in many species of plants and grains, including wheat, barley, corn, rice, and bamboo. Its presence in grains makes for large feedstocks where it is concentrated in the arabinoxylan of waste hulls and spent grain from fermentation brewing. Ferulic acid has long been recognized as a beneficial antioxidant in skin formulations [25-29] and is regarded as safe both topically applied as a dietary antioxidant. The UV stabilization of ferulic acid has also been established, where formulation with vitamins C & E increase their chemical stability during UV irradiation.

A limitation of ferulic acid is that it does not formulate into oils well and has limited water solubility. It exhibits slight lipophilicity, and can penetrate into the skin. This reduces the amount of protection ferulic acid can offer alone, as it should sit on the top of the skin to absorb the most UV radiation. In order to overcome these limitations, ferulic acid may be combined with larger molecules to enhance solubility in formulations, decrease skin penetration, and retain its photoprotective properties.

Research by Compton et al. has shown successful enzymatic transesterification of triglycerides, such as a vegetable oil, to make phytochemical-containing compositions (PCCs). For example, the enzymatic reaction of ethyl ferulate with triglyceride results in a mixture of products including unreacted starting materials (triglyceride oil, ethyl ferulate (EF)), the main reactant Feruloyl diacylglycerol (FDAG), and the minor reactants feruloyl monoacylglycerol (FMAG), Feruloyl glycerol (FG), Ferulic acid, and fatty acid ehyl esters wherein R represents-OH or a fatty acid C₂-C₂₂:

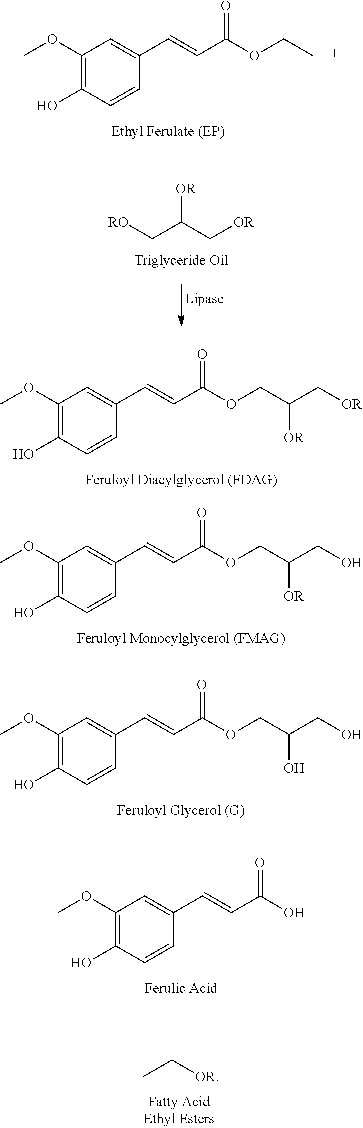

More generally the diacylglycerols (the primary constituent of the PCCs) are generally characterized by formula I:

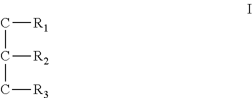

wherein at least two of the R₁, R₂, and R₃ are non-fatty acid carboxylates, and the other of R₁, R₂, and R₃, are either a C₂-C₂₄, fatty acid moiety, OH, or a non-fatty acid carboxylate wherein the first and second non-fatty acid carboxylates, when present, are the same or different. In such compositions, the first non-fatty acid carboxylate may comprise a first phytochemical and the second non-fatty acid carboxylate may comprise a second phytochemical, wherein the first and second phytochemicals are the same or different and may be of the type previously disclosed herein.

The PCC comprises a compound containing at least one UV-absorbing chromophore. Alternatively the PCC comprises a phytochemical. As used herein, “phytochemicals” are non-nutritive plant chemicals that have protective or disease preventive properties. Phytochemicals suitable for use in this disclosure may possess desirable characteristics such as for example UV absorbing properties, anti-aging properties, antibacterial, antineoplastic properties, antioxidant properties, antiviral or other photoactive, bioactive, or optical properties. In some cases, acyl esters of various phytochemicals are among the compounds that may be used in the present techniques. Examples of suitable phytochemicals include but are not limited to: flavonoids, isoflavones (phytoestrogens), isothio cyanates, organosulfur compounds, saponins, capsaicin, sterols, and particularly hydroxycinnamic acid derivatives such as coumaric, caffeic, chlorogenic, ferulic and sinapic acids.

In one embodiment, the phytochemical comprises any carboxyl containing phytochemical. Alternatively, the phytochemical comprises an aromatic species, an unsaturated isoprenoid, an unsaturated terpenoid, a hindered hydroxy-substituted cinnamic acid, an unhindered hydroxy-substi-tuted cinnamic acid or combinations thereof. In embodiments wherein the phytochemical comprises a hydroxy substituted cinnamic acid, the substituents may be located at positions 2, 3, 4, 5, 6 or combinations thereof. Examples of phytochemicals suitable for use in this disclosure include without limitation maleanilic acid, homovanillic acid, folic acid, crocetin coumaric acid, caffeic acid, ferulic acid, sinapic acid, derivatives thereof or combinations thereof. Useful properties attributed to those chemicals include antioxidant properties and anti-bacterial activity.

Compton et al. was able to attach ferulic acid onto triglycerides resulting in feruloyl-substituted or coumaryl-substituted acylglycerols (U.S. Pat. No. 6,346,236) and increasing their solubility in oil-based formulations^{65,66}. These compounds are generally characterized by formula I:

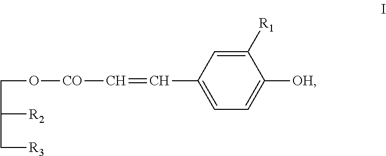

wherein R₁═OCH₃, or H; and wherein R₂ and R₃ are each independently selected from OH and a C₂-C₂₄ fatty acid moiety. Note, for ferulic acid, R₁ is OCH₃ and R₁ and R₂ are variable and dependent on the oils used. Different vegetable oils have different fatty acid chain lengths.

Further, their ability to act as antioxidants and photostabilizers is not inhibited once lipophilized.⁶⁷⁻⁷⁰

Microsilica. Other ingredients, such as micro silica, can be used to improve the texture and spreadability of the sunscreen. Microsilica, also known as fumed silica or colloidal silica, is a fine, amorphous, white powder that is widely used in various industrial applications. In recent years, microsilica has been studied for its potential use in sunscreens to boost SPF. Microsilica is capable of improving the sunscreen's coverage and enhancing its ability to protect the skin from the harmful effects of UV radiation [32, 33]. Microsilica is also considered to be safe for use in cosmetic products, as it is not absorbed into the skin and is not metabolized by the body.

Porous micro silica, refers to silica particles with an internal porous structure. These pores can vary in size and distribution, depending on the manufacturing process and intended application. Porous micro silica particles offer additional benefits compared to non-porous micro silica, such as increased surface area and enhanced adsorption capacity.

A mechanism by which microsilica enhances SPF is by improving the coverage of the active ingredients in the sunscreen. Microsilica particles are very small, which enables them to penetrate the skin's surface more deeply and fill in the gaps between the active ingredients. This improves the coverage of the sunscreen and increases its effectiveness in protecting the skin from UV radiation.

Another benefit of microsilica is its ability to scatter light. Microsilica particles are highly reflective, which helps to scatter the UV radiation and prevent it from penetrating the skin. This further enhances the sunscreen's ability to protect the skin from the harmful effects of UV radiation.

Blending microsilica with zinc oxide or titanium dioxide can increase the SPF of the sunscreen by improving the scattering and reflection of UV radiation. Microsilica has a high refractive index, which allows it to scatter UV radiation effectively. Studies have shown that the addition of microsilica to zinc oxide or titanium dioxide can increase the SPF of the sunscreen by a significant amount. For example, Park et al. (2013) found that adding microsilica to a ZnO-containing sunscreen increased the SPF by up to 45%, compared to the sunscreen without microsilica [34]. The authors attributed this effect to the improved adhesion of the ZnO particles to the skin, as well as the enhanced dispersion and stabilization of the ZnO particles in the formulation due to the microsilica. Similarly, Kumar et al. (2019) reported that incorporating microsilica into a TiO2-containing sunscreen increased the SPF by up to 26%, compared to the sunscreen without microsilica [35]. The authors suggested that the microsilica improved the film-forming properties and reduced the agglomeration of the TiO2 particles, leading to a more uniform and effective distribution on the skin.

The mechanisms underlying the SPF enhancement by microsilica in combination with ZnO or TiO₂ are not fully understood but may involve several factors. Microsilica can modify the rheological properties of the sunscreen formulation and improve the dispersion and stability of the metal oxide particles, thus enhancing their UV-blocking efficiency. Microsilica can also reduce the clumping and settling of the metal oxide particles during storage or use, which can lead to uneven application and lower SPF. Moreover, microsilica can form a film on the skin surface that can increase the adhesion and retention of the metal oxide particles, as well as provide a smoother and more uniform coverage.

Microsilica is typically added to sunscreens in concentrations of between 1% and 5% and the method of incorporating microsilica into the sunscreen is important [37-41]. Microsilica can be added to the oil phase or water phase of the sunscreen formulation. However, the microsilica must be well-dispersed throughout the formulation to achieve optimal coverage and UV radiation scattering. High shear mixing or ultrasonication can be used to achieve uniform dispersion of the microsilica throughout the formulation. The choice of emulsifier is also critical in formulating microsilica. An emulsifier is necessary to ensure that the oil and water phases of the sunscreen formulation are evenly distributed. Studies have shown that the type and concentration of the emulsifier can affect the stability of the microsilica in the formulation and the overall SPF of the sunscreen. Non-ionic emulsifiers such as polysorbates or sorbitan esters are commonly used in sunscreen formulations.

Finally, the compatibility of microsilica with other active ingredients in the sunscreen formulation should be considered. Microsilica has been shown to enhance the efficacy of some UV filters such as avobenzone, while it may reduce the efficacy of others such as octinoxate. Therefore, it is important to test the compatibility of microsilica with other active ingredients in the sunscreen formulation to ensure optimal SPF enhancement.

### UV-Filters and Other Molecules can Act as SPF Boosters

SPF boosters are UV filters or other ingredients that are added to sunscreens to increase their Sun Protection Factor (SPF). These ingredients work by enhancing the UV protection of the sunscreen, allowing it to provide greater protection against sunburn, skin aging, and skin cancer but do not necessarily posess the ability to have high SPF on their own. They can also help improve other physical properties of sunscreens. For example, when ZnO or TIO₂ are used at high concentrations they can leave a white cast on the skin and can be difficult to formulate into sunscreens. By adding an SPF booster the concentration of ZnO or TiO₂ can be reduced, reducing or eliminating white-cast and making the formulation easier to apply and feel better. Thus, SPF boosters are ingredients that are added to sunscreens to increase their UV protection and SPF. These ingredients work by enhancing the effectiveness of other UV filters or by providing additional UV protection. The use of SPF boosters can help to improve the performance and effectiveness of sunscreens, allowing them to provide better protection against sun damage and skin cancer.

Sunscreens play a crucial role in protecting the skin from harmful UV radiation, which can cause skin damage and increase the risk of skin cancer. The use of physical and organic sunscreens, UV filters, and SPF boosters can provide effective protection against UV radiation. However, there are challenges associated with the formulation of these ingredients in sunscreens, such as reducing whitecast and improving the stability of organic sunscreens. Novel approaches to sunscreen formulation, such as the use of nanoparticle sunscreens and antioxidant and anti-inflammatory ingredients, may offer safer and more effective alternatives to traditional sunscreen formulations.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indictates otherwise.

In some embodiments, percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

### Compositions

In some embodiments, formulations and compositions (these terms are used interchangeably herein) of the present disclosure incorporate feruloyl glycerides to increase SPF of the compositions.

In some embodiments, compositions contemplated that incorporate one or more feruloyl glycerides of the present disclosure include a sunscreen, a sun block, a makeup, a makeup remover composition, a lotion, a skin care product, and the like. In other embodiments, feruloyl glycerides of the present disclosure can be incorporated into paints, coatings, textiles, and other industrial applications.

In some embodiments, effective percentages of feruloyl glycerides of the present disclosure can range from about 0.5% to about 10%, or about 0.1% to about 20%, or about 0.05% to about 30%, or about 0.01% to about 50% or more of a contemplated composition. Other effective percentages contemplated herein are about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 95% of a contemplated composition.

Other ingredients (such as any of those described elsewhere herein) can each be incorporated into contemplated compositions in amounts of about 0.01-0.1%, or about 0.1-1%, or about 1-5%, or about 5-10%, or about 10-20%, or about 20-50%, or about 50-99.9%.

In some embodiments, contemplated formulations can be made using results of the initial testing incorporating feruloyl glycerides (for example, soy, coconut, or other compositions) of the examples (below) and other ingredients.

Ingredients that can be used in contemplated formulations include, for example, ingredients that serve particular functions within the formulations. For example, such ingredients include solvents, humectants, bases, emulsifiers, emollients, detergents, polymers, actives, silica, thickeners, and sunscreen agents. Amounts of such ingredients, to the extent any one of which is included can be varied, as desired to obtain intended formulations.

Other contemplated ingredients include active ingredients such as a retinoid, hyaluronic acid, vitamin C or a derivative thereof, vitamin D or derivative thereof, vitamin E or derivative thereof, a tocopherol, a bakuchiol, or a photosensitive active ingredient.

Specific, non-limiting examples of ingredients contemplated for use herein inclue water, propanediol (Zemea), tromethamine (Tris-amino Ultra PC, Angus), feruloyl glycerides (FSG-33, FCG-33, Midwest Bioprocessing Center), Carpylic/Capric triglycerides (Ethox), Sorbitan Laurate (Span 20, Croda), Bis-ocyldodecyl dimer dilionoleat/propanediol copolymer (Cosmosurf DDG-20, Surfatech), tri (polyglyceryl-3/lauryl) hydrogenated trilinoleate (Cithrol PGTL, Croda), silica (Soesphere H-33 or H-121, ACG or Sunspheres Bio SPF Booster, Dow), Zinc Oxide (Zinclear XP Powder, Antaria or ZnO Tayca, Mitsubishi).

In some embodiments, compositions contemplated herein can incorporate any ingredient desired in addition to those disclosed elsewhere herein.

Specific contemplated examples of formulations are disclosed in the tables below. Notes below the tables provide examples of formulating instructions.

TABLE 1
Sunscreen with 2.5% iActive feruloyl glycerides and 10% Zinclear XP
Powder.

| PHASE | % | INCI | FUNCTION |
|---|---|---|---|
| A | 52.4 | Water | Solvent |
| B | 10.0 | Propanediol | Humectant |
| B | 0.1 | Tromethamine | Bases |
| C | 2.5 | Feruloyl Glycerides | Actives |
| C | 17.5 | Caprylic/Capric Triglycerides | Emollients |
| C | 2.0 | Sorbitan Laurate | Spans/Tweens |
| C | 2.0 | Bis-Octyldodecyl Dimer Dilionoleate/ | Polymers |
| | | Propanediol Copolymer | |
| C | 1.5 | Tri(Polyglyceryl-3/Lauryl) Hydrogenated | Emulsifier |
| | | Trilinoleate | |
| D | 2.0 | Silica | Solesphere |
| D | 10.0 | Zinc Oxide | Sunscreen |

Premix B, then add to A with sharp-bladed impeller.
Premix C, then add D.
Slowly, add A/B to C/D with impeller.
Homogenize, if needed.

TABLE 2
Sunscreen with 5% iActive feruloyl glycerides and 10% Zinclear XP
Powder.

| PHASE | % | INCI | FUNCTION |
|---|---|---|---|
| A | 52.4 | Water | Solvent |
| B | 10.0 | Propanediol | Humectant |
| B | 0.1 | Tromethamine | Bases |
| C | 5.0 | Feruloyl Glycerides | Actives |
| C | 15.0 | Caprylic/Capric Triglycerides | Emollients |
| C | 2.0 | Sorbitan Laurate | Spans/Tweens |
| C | 2.0 | Bis-Octyldodecyl Dimer Dilionoleate/ | Polymers |
| | | Propanediol Copolymer | |
| C | 1.5 | Tri(Polyglyceryl-3/Lauryl) Hydrogenated | Emulsifier |
| | | Trilinoleate | |
| D | 2.0 | Silica | Solesphere |
| D | 10.0 | Zinc Oxide | Sunscreen |

Premix B, then add to A with sharp-bladed impeller.
Premix C, then add D.
Slowly, add A/B to C/D with impeller.
Homogenize, if needed.

TABLE 3
Sunscreen with 5% iActive feruloyl glycerides and 10% Zinclear XP
Powder and Dow Sunspheres.

| PHASE | % | INCI | FUNCTION |
|---|---|---|---|
| A | 52.4 | Water | Solvent |
| B | 10.0 | Propanediol | Humectant |
| B | 0.1 | Tromethamine | Bases |
| C | 5.0 | Feruloyl Glycerides | Actives |
| C | 15.0 | Caprylic/Capric Triglycerides | Emollients |
| C | 2.0 | Sorbitan Laurate | Spans/Tweens |
| C | 2.0 | Bis-Octyldodecyl Dimer Dilionoleate/ | Polymers |
| | | Propanediol Copolymer | |
| C | 1.5 | Tri(Polyglyceryl-3/Lauryl) Hydrogenated | Emulsifier |
| | | Trilinoleate | |
| D | 2.0 | Styrene/Acrylates Copolymer | Thickener |
| D | 10.0 | Zinclear XP Powder | Sunscreen |

Premix B, then add to A with sharp-bladed impeller.
Premix C, then add D.
Slowly, add A/B to C/D with impeller.
Homogenize, if needed.

TABLE 4
Sunscreen with 5% iActive feruloyl glycerides and 5% Zinclear XP.

| PHASE | % | INCI | FUNCTION |
|---|---|---|---|
| A | 52.4 | Water | Solvent |
| B | 10.0 | Propanediol | Humectant |
| B | 0.1 | Tromethamine | Bases |
| C | 5.0 | Feruloyl Glycerides | Actives |
| C | 20.0 | Caprylic/Capric Triglycerides | Emollients |
| C | 2.0 | Sorbitan Laurate | Spans/Tweens |
| C | 2.0 | Bis-Octyldodecyl Dimer Dilionoleate/ | Polymers |
| | | Propanediol Copolymer | |
| C | 1.5 | Tri(Polyglyceryl-3/Lauryl) Hydrogenated | Emulsifier |
| | | Trilinoleate | |
| D | 2.0 | Silica | Solesphere |
| D | 5.0 | Zinc Oxide | Sunscreen |

Premix B, then add to A with sharp-bladed impeller.
Premix C, then add D.
Slowly, add A/B to C/D with impeller.
Homogenize, if needed.

TABLE 5
Sunscreen with 5% iActive feruloyl glycerides and 10% Mitsubishi
ZnO.

| PHASE | % | INCI | FUNCTION |
|---|---|---|---|
| A | 53.4 | Water | Solvent |
| B | 10.0 | Propanediol | Humectant |
| B | 0.1 | Tromethamine | Bases |
| C | 5.0 | Feruloyl Coconut Glycerides | Actives |
| C | 15.0 | Caprylic/Capric Triglycerides | Emollients |
| C | 0.5 | Sorbitan Laurate | Spans/Tweens |
| C | 2.0 | Bis-Octyldodecyl Dimer Dilionoleate/ | Polymers |
| | | Propanediol Copolymer | |
| C | 2.0 | Tri(Polyglyceryl-3/Lauryl) Hydrogenated | Emulsifier |
| | | Trilinoleate | |
| D | 2.0 | Silica | Solesphere |
| D | 10.0 | Zinc Oxide (ZnO Tayca) | Sunscreen |

Premix B, then add to A with sharp-bladed impeller.
Premix C, then add D.
Slowly, add A/B to C/D with impeller.
Homogenize, if needed.

TABLE 6
Sunscreen with 5% iActive and 10% Zinclear XP Powder, with no
Solesphere.

| PHASE | % | INCI | FUNCTION |
|---|---|---|---|
| A | 55.4 | Water | Solvent |
| B | 10.0 | Propanediol | Humectant |
| B | 0.1 | Tromethamine | Bases |
| C | 5.0 | Feruloyl Coconut Glycerides | Actives |
| C | 15.0 | Caprylic/Capric Triglycerides | Emollients |
| C | 0.5 | Sorbitan Laurate | Spans/Tweens |
| C | 2.0 | Bis-Octyldodecyl Dimer Dilionoleate/ | Polymers |
| | | Propanediol Copolymer | |
| C | 2.0 | Tri(Polyglyceryl-3/Lauryl) Hydrogenated | Emulsifier |
| | | Trilinoleate | |
| D | 10.0 | Zinc Oxide (ZnO Taya) | Sunscreen |

Premix B, then add to A with sharp-bladed impeller.
Premix C, then add D.
Slowly, add A/B to C/D with impeller.
Homogenize, if needed.

TABLE 7
Sunscreen with 5% iActive feruloyl glycerides glycerides for
enhancement and SOLESPHERE H-33 for an SPF boost!

| PHASE | % | INCI | FUNCTION |
|---|---|---|---|
| A | 53.4 | Water | Solvent |
| B | 10.0 | Propanediol | Humectant |
| B | 0.1 | Tromethamine | Bases |
| C | 5.0 | Feruloyl Soy Glycerides | Actives |
| C | 15.0 | Caprylic/Capric Triglycerides | Emollients |
| C | 0.5 | Sorbitan Laurate | Spans/Tweens |
| C | 2.0 | Bis-Octyldodecyl Dimer Dilionoleate/ | Polymers |
| | | Propanediol Copolymer | |
| C | 2.0 | Tri(Polyglyceryl-3/Lauryl) Hydrogenated | Emulsifier |
| | | Trilinoleate | |
| D | 2.0 | Silica | Solesphere |
| D | 10.0 | Zinc Oxide | Sunscreen |

Premix B, then add to A with sharp-bladed impeller.
Premix C, then add D.
Slowly, add A/B to C/D with impeller.
Homogenize, if needed.

TABLE 8
Sunscreen with 5% iActive feruloyl glycerides, which is added during
the powder phase, and 10% Zinclear XP Powder.

| PHASE | % | INCI | FUNCTION |
|---|---|---|---|
| A | 52.4 | Water | Solvent |
| B | 10.0 | Propanediol | Humectant |
| B | 0.1 | Tromethamine | Bases |
| D | 1.5 | Tri(Polyglyceryl-3/Lauryl) Hydrogenated | Emulsifier |
| | | Trilinoleate | |
| C | 15.0 | Caprylic/Capric Triglycerides | Emollients |
| C | 2.0 | Sorbitan Laurate | Spans/Tweens |
| C | 2.0 | Bis-Octyldodecyl Dimer Dilionoleate/ | Polymers |
| | | Propanediol Copolymer | |
| D | 5.0 | Feruloyl Soy Glycerides | Actives |
| D | 2.0 | Silica | Solesphere |
| E | 10.0 | Zinc Oxide | Sunscreen |

Premix B, then add to A with sharp-bladed impeller.
Premix C.
Premix D, add to E.
Add D/E to C with impeller.
Slowly, add A/B to C/D/E.

### EXAMPLES

The Examples that follow are illustrative of specific embodiments of the disclosure, and various uses thereof. They are set forth for explanatory purposes only and should not be construed as limiting the scope of the disclosure in any way.

### Example 1. Assessing Formulation Properties of Feruloyl Glycerides in Silico

Using the Formulating for Efficacy® Software (ACT Solutions) a generalized SMILES sequence of feruloyl glycerides (COC1=CC(CCC(COCC(OC(CCCCCCC/C=C/CCCCCCCC)=O)COC(CCCCCCC/C=C/CCCC CCCC)=O)=O)=CC(O)=C1) was analyzed for solubility and absorption into the skin. This sequence conferred a triglyceride molecule with two fatty acid chains of linoleic acid and one ferulic acid moeity attached to the glycerol backbone and is a good representation for a variety of feruloyl glycerides. The results indicated that even if the molecules were fully dissolved in glycerin, none reaches the viable epidermis after 24 hours. This is likely due in part to the large molar volume of the molecules. The Active Skin Gap (ASG) is relatively high. These two factors greatly impact (the lack of) delivery into the skin. Active Skin Gap (ASG) measures diffusion to dermis and values of greater than 8 indicated that there are challenges for delivery to the dermis. Molar Volume (Mvol) measures diffusion into dermis and values of greater than 500 indicate the molecules do not easily penetrate skin. Organic sunscreen molecules tend to have a low molar volume AND low ASG and frequently have been seen to penetrate the skin and enter the bloodstream. This is suggested by the Formulating for Efficacy software. For example, Octisalate, Homosalate, and Octocrylene have Mvol calculated at 245.5, 246.0, and 341.3, respectively, and Active Skin Gap levels of 3.2, 4.0, and 5.7, respectively. This results in a calculation that octisalate is 90% delivered to the dermis after 24 hours and octocrylene is greater than 45% delivered to the dermis after 24 hours. On the other hand, feruloyl soy glycerides have a high Mvol value of 824 and active skin gap value of 9.9 and are essentially not delivered to the dermis after 24 hours exposure. As a result, these molecules seem best positioned as a UV booster and an antioxidant that work to stabilize other sensitive bioactives and lend oxidative stability on the surface of the skin, possibly protecting other actives as well. This also suggested utility in inorganic sunscreen formulations, both with TiO₂ and ZnO for daily and beach sun protection and suggested formulations to be tested.

### Example 2. Testing the Ability of Feruloyl Glycerides to Boost SPF with ZnO Formulations

In order to determine the novelty and efficacy of using feruloyl glycerides in typical cosmetic and sunscreen formulations, a variety of combinations of feruloyl glycerides (FG) were made. Two feruloyl glyceride ingredients of interest were tested: feruloyl soy glycerides (iActive FSG-33, Midwest Bioprocessing Center) and feruloyl coconut glycerides (iActive FCG-33, Midwest Bioprocessing Center). The feruloyl glycerides were added in varying amounts to a variety of different sunscreen formulations to test their effect on SPF. In addition to a sunscreen ingredient such as ZnO, porous micro silica spheres were also tested (solesphere H-33, AGC Chemicals). One main formulation was used where all variables were kept constant except the level of feruloyl glycerides (Coconut and Soy).

To carry out these tests, in vitro SPF levels were determined. In vitro levels can differ from in vivo test results on humans but are easier to control, provide good correlation between relative SPF values of compositions, and are easier and faster to perform. For these reasons, they are an industry standard test for new formulations. In vitro SPF level testing was performed by the University of Toledo using a Labsphere 2000, the standard analytical instrument for carrying out the tests using the method described in ISRN Dermatol 2012; 2012:352135.

Typical formulas using 5% feruloyl glycerides (0-10% tested), and 10% Zinc oxide (0-20% tested) are shown in the formulations below:

TABLE 9
5% Coconut iActive and 10% Zinc Oxide.

| Phase | Tradename | INCI Name | Function | Grams | % |
|---|---|---|---|---|---|
| A | Water | Water | Solvent | 52.4 | 52.4 |
| B | Zemea | Propanediol | Humectant | 10 | 10 |
| B | Tris-amino | Tromethamine | Base | 0.1 | 0.1 |
| | Ultra PC | | | | |
| C | Feruloyl | Coconut oil ethyl | Active | 5 | 5 |
| | Coconut | ferulate esters | | | |
| | Glyceride | | | | |
| C | Caprylic/Capric | Caprylic/Capric | Emollient | 15 | 15 |
| | Triglycerides | Triglycerides | | | |
| C | Span 20 | Sorbitan Laurate | Span | 2 | 2 |
| C | Cosmosurf | Bis-Octyldodecyl | Polymer | 1.5 | 1.5 |
| | DDG-20 | Dimer Dilionoleate | | | |
| | | Propanediol | | | |
| | | Copolymer | | | |
| C | Cithrol PGTL | Tri(Polyglcyeryl- | Emulsifier | 1.5 | 1.5 |
| | | 3/Lauryl) | | | |
| | | Hydrogenated | | | |
| | | Trilinoleate | | | |
| D | Solesphere H- | Silica | Active | 2 | 2 |
| | 33 | | | | |
| D | Zinclear XP | Zinc Oxide | Sunscreen | 10 | 10 |
| | Powder | | | | |
| | | | TOTAL | 99.5 | 99.5 |

Procedure:
1) Premix B, then add to A with sharp-bladed impeller
2) Premix C, then add D
3) Add A/B to C/D with impeller

TABLE 10
5% Soy iActive and 10% Zinc Oxide.

| Phase | Tradename | INCI Name | Function | Grams | % |
|---|---|---|---|---|---|
| A | Water | Water | Solvent | 53.4 | 53.4 |
| B | Zemea | Propanediol | Humectant | 10 | 10 |
| B | Tris-amino Ultra | Tromethamine | Base | 0.1 | 0.1 |
| | PC | | | | |
| C | Feruloyl Soy | Feruloyl soy | Active | 5 | 5 |
| | Glyceride | glycerides | | | |
| C | Caprylic/Capric | Caprylic/Capric | Emollient | 15 | 15 |
| | Triglycerides | Triglycerides | | | |
| C | Span 20 | Sorbitan Laurate | Span | 0.5 | 0.5 |
| C | Cosmosurf | Bis-Octyldodecyl | Polymer | 2 | 2 |
| | DDG-20 | Dimer Dilionoleate | | | |
| | | Propanediol | | | |
| | | Copolymer | | | |
| C | Cithrol PGTL | Tri(Polyglcyeryl- | Emulsifier | 2 | 2 |
| | | 3/Lauryl) | | | |
| | | Hydrogenated | | | |
| | | Trilinoleate | | | |
| D | Solesphere H-33 | Silica | Active | 2 | 2 |
| D | Zinclear XP | Zinc Oxide | Sunscreen | 10 | 10 |
| | Powder | | | | |
| | | | TOTAL | 100 | 100 |

Procedure:
1) Premix B, then add to A with sharp-bladed impeller
2) Premix C, then add D
3) Add A/B to C/D with impeller

The results are outlined in Table 11 and Table 12. It can be seen that on their own, the feruloyl glycerides give only low levels of SPF with feruloyl coconut glycerides giving an in vitro SPF of 3.4 (test 23498) and feruloyl soy glycerides giving an in vitro SPF of 3.0 (test 23500). In both of these formulations, critical wavelengths of 346.7 nm (Coconut) and 346.6 nm (soy) were below the threshold of 370 desired for broad spectrum sunscreens. The critical wavelength (CW) is the wavelength at which the sunscreen allows 10% of the rays to penetrate. A sunscreen with a critical wavelength over 370 nm is considered by the FDA to provide excellent UVA protection A 10% ZnO load typically gave an SPF of 20. When 10% ZnO and 5% feruloyl glycerides were used, the in vitro SPF value was determined to be 30.9 (test 23810) for feruloyl coconut glycerides and 40.5 (test 23813) for feruloyl soy glycerides and the critical wavelengths were 375.7 and 375.3, respectively.

Based on this there appeared to be some unexpected synergistic interactions between ZnO and feruloyl glycerides. These results demonstrate that adding the tested feruloyl glycerides to an inorganic sunscreen active leads to a nonadditive and surprising boost in SPF despite the low intrinsic value of the feruloyl glycerides themselves.

### Example 3. Additional Testing of Feruloyl Glycerides with ZnO and Porous Microsilica

Porous microsilica has been shown to provide a modest boost to sunscreen formulations. In an initial test using 20% feruloyl glycerides (FG) and 10% ZnO, an extremely large SPF 285.4, 372 nm CW was achieved, whereas the sunscreen emulsion with no FG (just Caprylic/capric Triglyceride) got an SPF reading of 21.4 and CW of 375.0 nm.

Porous micro silica occasionally doubles SPF but again, is generally lower. Still, an SPF of 285.4 when combined with feruloyl glycerides while still passing the critical wavelength threshold of 370 nm is unusual and surprising. In addition, the formulation objectively feels nice and does not leave the skin with a white cast.

Using this information additional formulations were made. A formulation with 5% feruloyl coconut glycerides (FCG) and 10% Zinc Oxide (ZnO) yielded an in vitro SPF of 156.6 and a Critical Wavelength of 371.7 nm. The feruloyl soy glycerides also gave similar results. This information supported the theory that feruloyl glycerides are making ZnO work much more effectively, lending credence to the notion that there is a lot of energy dissipation that is going on at the surface of ZnO and that feruloyl glycerides aided this. It also appeared that there may be a complex interaction between the uncoated ZnO, the porous spherical silica, and the FG.

After additional testing, it seemed that there is likely a special relationship between the feruloyl glycerides and the ZnO as well as the feruloyl glycerides and the porous micro silica. Formulations were also made using 10% Mitsubishi uncoated ZnO in place of Antaria ZinClear XP Powder and got somewhat lower but still good in vitro numbers of SPF 62.3/371.1 nm CW.

TABLE 11
Initial Results.

| | | | | Solesphere | | |
|---|---|---|---|---|---|---|
| 5 Digit | Formulation | % feruloyl | ZnO | Porous Micro | In-vitro | Critical |
| Number | Type | glyceride | % | silica % | SPF | Wavelength |
| 23645 | W/O | 0 | 10% | 2% | 21.4 | 375.0 |
| 23498 | O/W | 5% Coconut | 0% | 0% | 3.4 | 346.7 |
| 23649 | W/O | 5% Coconut | 10% | 2% | 156.6 | 371.7 |
| 23800 | W/O | 2.5% Coconut | 10% | 2% | 25.4 | 374.5 |
| 23649 | W/O | 5% Coconut | 10% | 2% | 285.4 | 372.9 |
| 23500 | O/W | 5% Soy | 0% | 0% | 3.0 | 346.6 |
| 23811 | W/O | 5% Soy | 10% | 2% | 156.6 | 371.7 |
| 23822 | W/O | 2.5% Soy | 10% | 2% | 43.3 | 374.0 |

TABLE 12
Expanded Results.

| | | | | Solesphere | | |
|---|---|---|---|---|---|---|
| 5 Digit | Formulation | % feruloyl | ZnO | Porous Micro | In-vitro | Critical |
| Number | Type | glyceride | % | silica % | SPF | Wavelength |
| 23649 | W/O | 5% Coconut | 10% | 2% | 156.6 | 371.7 |
| 23800 | W/O | 2.5% Coconut | 10% | 2% | 25.4 | 374.5 |
| 23851 | W/O | 5% Coconut | 10% | 2% | 36.4 | 376.9 |
| | | | | (Sunspheres) | | |
| 23799 | W/O | 5% Coconut | 5% | 2% | 25.1 | 368.4 |
| 23823 | W/O | 5% Coconut | 10% | 2% | 62.3 | 371.1 |
| | | | (MZ) | | | |
| 23810 | W/O | 5% Coconut | 10% | 0% | 30.9 | 375.7 |
| 23811 | W/O | 5% Soy | 10% | 2% | 156.6 | 371.7 |
| 23822 | W/O | 2.5% Soy | 10% | 2% | 43.3 | 374.0 |
| 23815 | W/O | 5% Soy | 10% | 2% | 66.4 | 373.1 |
| | (premix) | | | | | |
| 23850 | W/O | 5% Soy | 10% | 2% | 36.6 | 376.9 |
| | | | | (Sunspheres) | | |
| 23812 | W/O | 5% Soy | 5% | 2% | 13.7 | 268.2 |
| 23816 | W/O | 5% Soy | 10% | 2% | 62.3 | 371.1 |
| | | iActive | (MZ) | | | |
| 23813 | W/O | 5% Soy | 10% | 0% | 40.5 | 375.3 |
| | | iActive | | | | |

All Zinc Oxide formulations made with Antaria Zinclear except MZ made with Mitsubishi Zinc Oxide. All TiO₂ obtained from Mitsubishi.

TABLE 13
TiO₂ and Organic Sunscreens.

| | | | | Solesphere | | |
|---|---|---|---|---|---|---|
| | | % | Sunscreen | Porous | In- | |
| 5 Digit | Formulation | feruloyl | Ingredient | Micro | vitro | Critical |
| Number | Type | glyceride | % | silica % | SPF | Wavelength |
| 24832 | W/O | 5% Soy | 15% TiO₂ | 2% | 69.5 | 371.3 |
| 24984 | W/O | 0% | 15% TiO₂ | 2% | 25.5 | 372 |
| 24687 | W/O | 5% Soy | Homosalate | 17% | 417 | 380 |
| | | | (15%), | | | |
| | | | octisalate | | | |
| | | | (5%), | | | |
| | | | octocrylene | | | |
| | | | (10%), | | | |
| | | | avobenzone | | | |
| | | | (3%). | | | |
| 25038 | W/O | 0 | Homosalate | 17% | 229.7 | 376.8 |
| | | | (15%), | | | |
| | | | octisalate | | | |
| | | | (5%), | | | |
| | | | octocrylene | | | |
| | | | (10%), | | | |
| | | | avobenzone | | | |
| | | | (3%). | | | |
| 24865 | W/O | 2.3% Coconut | 5% MT-150EX | 2% | 326.6 | 384 |
| | | | (TiO₂/AlOH/ | | | |
| | | | Stearic Acid) | | | |
| | | | 10% MT-700Z | | | |
| | | | (TiO₂/AlOH/ | | | |
| | | | Stearic Acid) | | | |
| 25107 | W/O | 0 | 5% MT-150EX | 2% | 234.0 | 384.3 |
| | | | (TiO₂/AlOH/ | | | |
| | | | Stearic Acid) | | | |
| | | | 10% MT-700Z | | | |
| | | | (TiO₂/AlOH/ | | | |
| | | | Stearic Acid) | | | |
| 23887 | O/W | 5% Soy | ZnO (2%, MZ) + | 2% (H- | 32.3 | 372.3 |
| | | iActive | TiO₂ (5%) | 121) | | |

MZ: Mitsubishi ZnO, MT: Mitsubishi TiO2. Homosalate, octisalate, and octocrylene obtained from Merck KGaA. Avobenzone obtained from DSM.

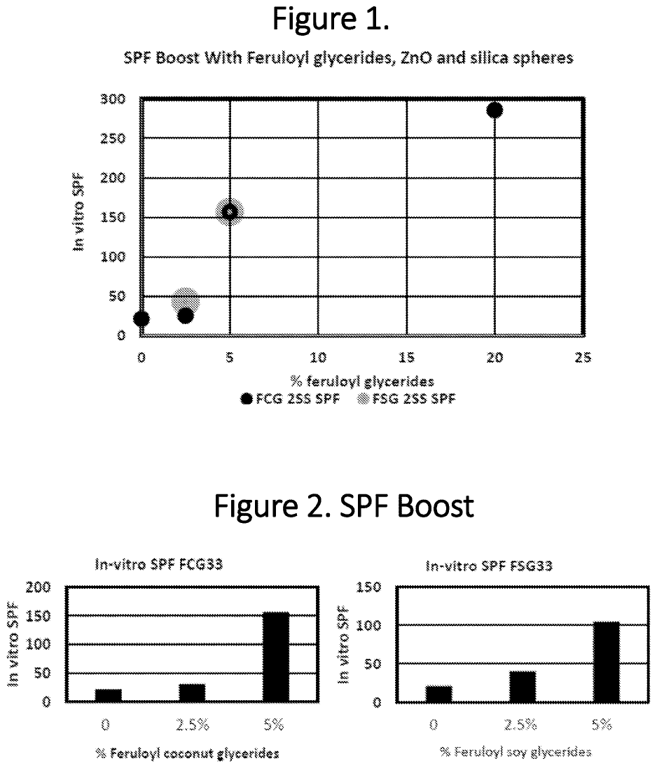

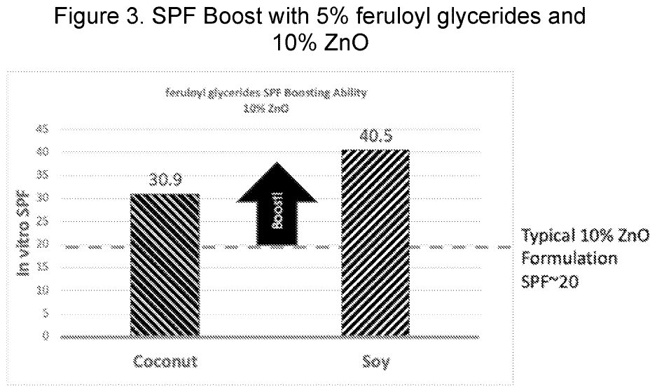

The data can effectively be seen in FIGS. 1, 2, and 3. FIG. 1 is a scatter plot showing that when either feruloyl coconut glycerides or feruloyl soy glycerides are combined with a mixture of ZnO and porous micro silica they have a significant boost in the overall SPF of the formulation. Likewise, the bar chart in FIG. 2 also shows that when either feruloyl coconut glycerides or feruloyl soy glycerides are combined with a mixture of ZnO and porous micro silica they have a significant boost in the overall SPF of the formulation. FIG. 3 shows the boost effect without the porous micro silica.

The data strongly suggest that when feruloyl glycerides are combined with a sunscreen agent such as ZnO or TiO₂ and/or porous micro silica or dispersion agents, the effect in increseasing SPF is multiplicative not just additive. This allows a number of improvements to the formulation of sunscreens and formulas designed to protect molecules from photodegradation. It allows a more transparent sunscreen to be developed since ZnO and/or TiO₂ levels can be significantly reduced to achieve similar SPFs.

Additional tests were run with a larger variety of conditions including using Sunspheres (Dow) instead of Solesphere and H-121 Solespheres as indicated in Table 12. These data reinforce and confirm the original results in Tables 11-13.

These results confirmed the repeatability of the data and showed that by adding feruloyl glycerides, a significant boost in in vitro SPF can be received. Of note is the O/W formulation with a combination of water dispersible TiO₂ and ZnO in the oil phase. This combination gave a good boost from a base of 22 (not shown) to over 30 (23887).

### Example 4. Formulating Feruloyl Glycerides with Titanium Dioxide or Organic Sunscreens

Table 3 shows the ability of the feruloyl glycerides to boost SPF when combined with another physical sunscreen ingredient, TiO₂, and with organic sunscreens. In a 15% mixture of TiO₂ with porous microsilica an in vitro SPF of 25.5 was achieved (24984). When 5% feruloyl soy glycerides were added, the SPF increased significantly to 69.5 (24832). Likewise when a mixture of organic sunscreens (homosalate, octisalate, octocrylene and avobenzone) to their maximum allowed amounts and 17% porous micro silica gave an in vitro SPF of 229 (25038). When 5% feruloyl soy glycerides were added the SPF was boosted to 417.

These data show the ability of feryloyl glycerides to boost SPF for a wide range of inorganic and organic sunscreen actives.

All publications, patents, and patent applications mentioned in this specification are herein incorporated by reference to the same extent as if each individual publication, patent, and patent application was specifically and individually indicated to be incorporated by reference.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

### REFERENCES

1. Gaspar, L. R. and P. M. Campos, Photostability and efficacy studies of topical formulations containing UV-filters combination and vitamins A, C and E. Int J Pharm, 2007. 343 (1-2): p. 181-9.
2. Temova Rakusa, Z., et al., Retinoid stability and degradation kinetics in commercial cosmetic products. J Cosmet Dermatol, 2021. 20 (7): p. 2350-2358.
3 Gianeti, M. D., et al., Benefits of combinations of vitamin A, C and E derivatives in the stability of cosmetic formulations. Molecules, 2012. 17 (2): p. 2219-30.
4. Allwood, M. C. and H. J. Martin, The photodegradation of vitamins A and E in parenteral nutrition mixtures during infusion. Clin Nutr, 2000. 19 (5): p. 339-42.
5 Drott, P., S. Meurling, and L. Meurling, Clinical adsorption and photodegradation of the fatsoluble vitamins A and E. Clin Nutr, 1991. 10 (6): p. 348-51.
6 Caffieri, S., et al., UVB photolysis of hydrocortisone 21-acetate. J Pharm Biomed Anal, 2008. 47 (4-5): p. 771-7.
7. Khattak, S. U., et al., In vitro evaluation of betamethasone esters for phototoxic potential. Drug Chem Toxicol, 2012. 35 (1): p. 43-7.
8. Khattak, S. U., et al., Photodegradation and stabilization of betamethasone-17 valerate in aqueous/organic solvents and topical formulations. AAPS PharmSciTech, 2013. 14 (1): p. 177-82.
9. Miolo, G., et al., UVB photolysis of betamethasone and its esters: characterization of photoproducts in solution, in pig skin and in drug formulations. J Photochem Photobiol B, 2009. 96 (1): p. 75-81.
10. Kryczyk, A., P. Zmudzki, and U. Hubicka, Determination of itraconazole and its photodegradation products with kinetic evaluation by ultra-performance liquid chromatography/tandem mass spectrometry. Biomed Chromatogr, 2016. 30 (11): p. 1733-1743.
11. Nardi, G., et al., Generation of Reactive Aryl Radical Intermediates in the Reductive Photodehalogenation of Itraconazole. RSC Adv, 2013. 4: p. 2687-2693.
12. Bartsch, H., A. Eiper, and H. Kopelent-Frank, Stability indicating assays for the determination of piroxicam—comparison of methods. J Pharm Biomed Anal, 1999. 20 (3): p. 531-41.
13. Jimenez, J. J., et al., Forced and long-term degradation assays of tenoxicam, piroxicam and meloxicam in river water. Degradation products and adsorption to sediment. Chemosphere, 2018. 191: p. 903-910.
14. Sammartino, M. P., et al., Photostability and toxicity of finasteride, diclofenac and naproxen under simulating sunlight exposure: evaluation of the toxicity trend and of the packaging photoprotection. Chem Cent J, 2013. 7 (1): p. 181.
15. Brisaert, M. G., I. Everaerts, and J. A. Plaizier-Vercammen, Chemical Stability of Tretinoin in Dermatological Preparations. Pharm. Acta Helv., 1995. 70: p. 161-166.
16. Martin, B., et al., Chemical stability of adapalene and tretinoin when combined with benzoyl peroxide in presence and in absence of visible light and ultraviolet radiation. Br J Dermatol, 1998. 139 Suppl 52: p. 8-11.
17. Tashtoush, B. M., E. L. Jacobson, and M. K. Jacobson, UVA is the major contributor to the photodegradation of tretinoin and isotretinoin: Implications for development of improved pharmaceutical formulations. Int J Pharm, 2008. 352 (1-2): p. 123-8.
18. Tolba, M. M. and R. M. El-Gamal, Determination of adapalene in gel formulation by conventional and derivative synchronous fluorimetric approaches. Application to stability studies and in vitro diffusion test. Chem Cent J, 2016. 10: p. 33.
19. Wang, S. Q. and H. W. Lim, Highlights and implications of the 2019 proposed rule on sunscreens by the US Food and Drug Administration. J Am Acad Dermatol, 2019. 81 (2): p. 650-651.
20. US Food and Drug Administration. Proposed Rule: Sunscreen Drug Products for Over-the-Counter Human Use. February 2019. (www.fda.gov/drugs/otc-drug-products/sunscreen-drug-products-over-counter-human-use).
21. Sharma, S. and S. Sharma, Sunscreens: A review on formulations and regulations. International Journal of Research in Pharmaceutical Sciences, 2020. 11: p. 429-437.
22. Draelos, Z. D., Sunscreens: Are they beneficial for health? An overview of endocrine disrupting properties of UV-filters. International Journal of Women's Dermatology, 2021. 7 (1): p. 54-59.
23. Wang, S. Q., Sunscreens in the United States: Current status and future outlook. Advances in Dermatology, 2019. 35: p. 115-123.
24. Lim, H. W and et al, Sunscreens: FDA regulation, and environmental and health impact. Photochemistry and Photobiology, 2021. 97 (2): p. 369-379.
25. Graf, E., Antioxidant potential of ferulic acid. Free Radic Biol Med, 1992. 13 (4): p. 435-448
26. Kumar, N. and V. Pruthi, Potential applications of ferulic acid from natural sources. Biotechnol Rep (Amst), 2014. 4: p. 86-93.
27. Raj, N. D. and D. A. Singh, A Critical Appraisal on Ferulic Acid: Biological Profile, Biopharmaceutical Challenges and Nano Formulations. Health Sci. Rev, 2022. 5: p. 100063.
28. Saija, A., et al., In vitro and in vivo evaluation of caffeic and ferulic acids as topical photoprotective agents. Int J Pharm, 2000. 199 (1): p. 39-47.
29. Srinivasan, M., A. R. Sudheer, and V. P. Menon, Ferulic Acid: therapeutic potential through its antioxidant property. J Clin Biochem Nutr, 2007. 40 (2): p. 92-100.
30. Compton, D. L., et al., Protection of Antioxidants, Vitamins E and C, from Ultraviolet Degradation Using Feruloylated Vegetable Oil. J. Am. Oil Chem. Soc., 2019. 96 (9): p. 999-1009.
31. Zhang, L. W., et al., A comparison of skin delivery of ferulic acid and its derivatives: evaluation of their efficacy and safety. Int J Pharm, 2010. 399 (1-2): p. 44-51.
32. Tadros, T. F., Emulsion Formation and Stability. 2013: John Wiley & Sons, Ltd.
33. Lin CW and et al., Sunscreen Compositions Containing Microsilica and Metal Oxide UV Filters. 2019: US.
34. Park, H., et al., Sunscreen composition comprising microsilica and zinc oxide. 2013: US.
35. Kumar, P., A. K. Mishra, and S. B. Mishra, Development of sunscreen cream with enhanced sun protection factor using microsilica-titanium dioxide hybrid particles. Journal of Cosmetic Science, 2019. 70 (1): p. 37-50.
36. Wu, J. and W. Liu, Recent advances in sunscreen nanotechnology. Nanomaterials, 2020. 10(6): p. 1066.
37. Liu G and et al., Influence of colloidal silica on the performance of sunscreen. Journal of Cosmetic Science, 2015. 66 (1): p. 47-56.
38. Kim H and et al., The efficacy and safety of colloidal silica as a UV absorber in sunscreen. Journal of Cosmetic Science., 2013. 64 (5): p. 347-358.
39. Li C and et al., Improvement of water resistance and ultraviolet protection of sunscreen cream by colloidal silica. Journal of Cosmetic Science, 2012. 63 (6): p. 397-408.
40. A., B., et al., and 2009;, Effect of fumed silica on the stability and SPF of O/W emulsions containing avobenzone. Journal of Cosmetic Science, 2009. 60 (5): p. 569-577.
41. Kim M and et al., Effects of emulsifiers on the physicochemical properties and in vitro skin permeation of sunscreens containing microsilica. International Journal of Cosmetic Science, 2019. 41 (4): p. 378-386.
42. Sarveiya, V., Risk, S., Benson, H. A., & Eadie, L. (2004). Liquid chromatographic assay for common sunscreen agents: application to in vivo assessment of skin penetration and systemic absorption in human volunteers. Journal of Chromatography B, 803 (2), 225-231.
43. Pelizzo M, Zattra E, Nicolosi P, Peserico A, Garoli D, Alaibac M. In vitro evaluation of sunscreens: an update for the clinicians. ISRN Dermatol. 2012; 2012:352135. doi: 10.5402/2012/352135. Epub 2012 Nov. 27. PMID: 23227355; PMCID: PMC3514825.
44. Nohynek, G. J., Antignac, E., Re, T., Toutain, H., Bakker, M. I., & Fernandez, M. F. (2019). Safety assessment of personal care products/cosmetics and their ingredients. Toxicology and applied pharmacology, 378, 114704.
45. Kornhauser, A., Coelho, S. G., Hearing, V. J., & editors. (2019). Sunscreens: regulations and commercial development. CRC Press.
46. Sayre, R. M., Agin, P. P., LeVee, G. J., & Marlowe, E. (1992). A comparison of in vivo and in vitro testing of sunscreening formulas. Photochemistry and photobiology, 56 (4), 573-578.
47. Wang, S. Q., Osterwalder, U., & Jung, K. (2019). Ex vivo evaluation of sun protection factor and protection against UVA-induced darkening by popular sunscreens. Journal of the American Academy of Dermatology, 81 (5), 1275-1280.
48. Smijs, T. G. and S. Pavel (2011). “Titanium dioxide and zinc oxide nanoparticles in sunscreens: focus on their safety and effectiveness.” Nanotechnol Sci Appl 4:95-112.

We claim:

1. A composition, comprising:
a) at least one sunscreen active agent; and
b) a compound of formula I: 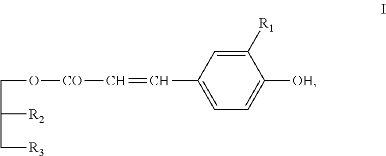
wherein R₁ is OCH₃ or H, and
wherein R₂ and R₃ are each independently selected from OH and a C₂-C₂₄ fatty acid moiety.

2. The composition of claim 1, wherein the at least one sunscreen active agent is a physical sunscreen comprising zinc oxide or titanium dioxide.

3. The composition of claim 1, wherein the at least one sunscreen active agent is an organic sunscreen comprising one or more of homosalate, octisalate, octocrylene, avobenzone, anthranilates, cinnamic derivatives, dibenzoylmethane derivatives, salicylic derivatives, camphor derivatives, triazine derivatives, benzophenone derivatives, B,β-diphenylacrylate derivatives, benzo-benzalmalonate benzimidazole derivatives, imidazolines, bisbenzoazolyl derivatives, p-aminobenzoic acid (PABA) derivatives, methylenebishydroxyphenylbenzotriazole derivatives, screening polymers and screening silicones, dimers derived from a-alkylstyrene, 4,4-diarylbutadienes and their mixtures.

4. The composition of any one of claims 1-3, wherein the combination of the at least one sunscreen active agent and the compound provide an SPF greater than the additive properties of the individual agents.

5. The composition of any one of claims 1-4, wherein the composition has an in vitro SPF of at least 10.

6. The composition of claim 2, wherein the combination is combined with a reduced amount of physical sunscreen to the point of yielding a formulation that is transparent and free of white cast effect while retaining high SPF.

7. A composition, comprising:
a) at least one sunscreen active agent;
b) a porous micro silica; and
c) a compound of formula I: 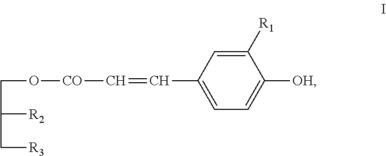
wherein R₁ is OCH₃ or H, and
wherein R₂ and R₃ are each independently selected from OH and a C₂-C₂₄ fatty acid moiety.

8. The composition of claim 7, wherein the combination of ingredients is combined with at least one physical or organic sunscreen.

9. The composition of claim 7 or 8, wherein the combination of ingredients provides an SPF greater than the additive properties of the individual agents.

10. The composition of claim 8, wherein the at least one physical sunscreen is one or more of zinc oxide or titanium dioxide.

11. The composition of claim 8, wherein the at least one organic sunscreen is one or more of homosalate, octisalate, octocrylene, avobenzone, anthranilates, cinnamic derivatives, dibenzoylmethane derivatives, salicylic derivatives, camphor derivatives, triazine derivatives, benzophenone derivatives, B,β-diphenylacrylate derivatives, benzo-benzalmalonate benzimidazole derivatives, imidazolines, bisbenzoazolyl derivatives, p-aminobenzoic acid (PABA) derivatives, methylenebishydroxyphenylbenzotriazole derivatives, screening polymers and screening silicones, dimers derived from a-alkylstyrene, 4,4-diarylbutadienes and their mixtures.

12. The composition of any one of claims 7-11, wherein the composition has an in vitro SPF of at least 10.

13. A composition, comprising:
a) an optional solvent;
b) an optional humectant;
c) an optional base;
d) a feruloyl glyceride;
e) an optional emollient;
f) an optional detergent;
g) an optional polymer;
h) an optional emulsifier;
i) an optional micro silica
j) an optional active; and
k) an optional sunscreen.

14. The composition of claim 13, comprising:
a) about 80-99.9% solvent; and
b) about 0.1% to about 20% of a feruloyl glyceride.

15. The composition of claim 13, comprising:
a) about 0.1% to about 20% of a feruloyl glyceride; and
b) about 1% to about 25% of a sunscreen.

16. The composition of claim 13, comprising:
a) about 0.1% to about 20% of a feruloyl glyceride;
b) about 1% to about 25% of a sunscreen; and
c) about 0.5% to about 20% of a micro silica.

17. The composition of claim 13, 15, or 16, wherein the sunscreen is a physical sunscreen comprising zinc oxide and/or titanium dioxide.

18. The composition of claim 13, 15, or 16, wherein the sunscreen is an organic sunscreen comprising one or more of homosalate, octisalate, octocrylene, avobenzone, anthranilates, cinnamic derivatives, dibenzoylmethane derivatives, salicylic derivatives, camphor derivatives, triazine derivatives, benzophenone derivatives, B,β-diphenylacrylate derivatives, benzo-benzalmalonate benzimidazole derivatives, imidazolines, bisbenzoazolyl derivatives, p-aminobenzoic acid (PABA) derivatives, methylenebishydroxyphenylbenzotriazole derivatives, screening polymers and screening silicones, dimers derived from a-alkylstyrene, 4,4-diarylbutadienes and their mixtures.

19. The composition of claim 13, comprising:
a) about 0.1% to about 20% of a feruloyl glyceride; and
b) an active ingredient comprising one or more of a retinoid, hyaluronic acid, vitamin C or a derivative thereof, vitamin D or derivative thereof, vitamin E or derivative thereof, a tocopherol, a bakuchiol, or a photosensitive active ingredient.

20. A topical composition, comprising:
a compound of formula I: 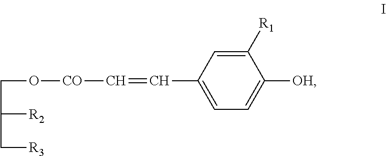
wherein R₁ is OCH₃ or H, and
wherein R₂ and R₃ are each independently selected from OH and a C₂-C₂₄ fatty acid moiety and the calculated active skin gap (AKG) is greater than 8 and Mvol is greater than 500 indicating that the molecule does not penetrate the skin.

21. The composition of claim 20, comprising:
a) about 0.1% to about 20% of a feruloyl glyceride.
